(19) United States
(12) Patent Application Publication
(10) Pub. No.: US 2024/0206927 A1
(43) Pub. Date: Jun. 27, 2024

(54) ORTHOPEDIC IMPLANT WITH NON-HELICAL FASTENER HOLE

(71) Applicant: Skeletal Dynamics, Inc., Miami, FL (US)

(72) Inventors: Jorge L. Orbay, Miami, FL (US); Robert Sixto, Miami, FL (US); Juergen Kortenbach, Miami, FL (US)

(21) Appl. No.: 18/544,866

(22) Filed: Dec. 19, 2023

Related U.S. Application Data
(60) Provisional application No. 63/476,507, filed on Dec. 21, 2022.

(51) Int. Cl. A61B 17/80; A61B 17/86

(52) U.S. Cl. CPC A61B 17/8052; A61B 17/8605

(57) ABSTRACT

An orthopedic implant adapted for fastening to a bone by use of a screw, the implant comprising a plate composed of a biocompatible material and a hole defining an internal hole wall and having an innermost surface; the hole adapted to receive said screw; the internal hole wall comprising a primary inner width and a secondary outer width with a distance therebetween defining a volume; the volume comprising a biocompatible material and a plurality of blind holes extending from the primary inner width to the secondary outer width resulting in a porosity; wherein the internal hole wall comprises a non-helical pattern. In at least one embodiment, 30% to 70% of the volume is comprised of the biocompatible material and the remaining 70% to 30% of the volume is devoid of any material. In another embodiment, the porosity of the volume is between 0.3 and 0.7.

### CLAIM OF PRIORITY

This application is being filed as a non-provisional patent application under 35 U.S.C. § 111(a) and 37 CFR § 1.53(b). This application claims priority under 35 U.S.C. § 119(e) to U.S. provisional patent application Ser. No. 63/476,507 filed on Dec. 21, 2022, the contents of which are incorporated herein by reference.

### FIELD OF INVENTION

The invention relates generally to orthopedic implants for fixation of bones, and in particular to bone plates having non-helical screw holes adapted to receive off-axis locking bone screws.

### BACKGROUND OF THE INVENTION

Orthopedic bone plates are widely used to correct all kinds of bone defects and trauma, such as bone fractures. Bone plates are sometimes affixed to a bone by means of screws. Such plates have openings through which a screw is inserted so that it can penetrate the underlying bone. Broadly speaking, there are two types of plate-to-screw interfaces—locking and non-locking.

With a non-locking bone-plate interface, the interior surface of the plate hole is generally smooth, as is the head of the screw. Although the shaft of the screw is threaded, the head of the screw is not. The head of a non-locking screw is either larger in diameter than the diameter of the hole, or has some other feature, such as a conical profile, which prevents the screw from completely penetrating the hole. As the screw is threaded into the bone, the oversized head, or conical profile, begins to compress the plate against the bone as the screw gains “purchase” into the bone. It is this compression that holds the plate to the bone. However, the screw is not locked with respect to the plate.

With a locking bone-plate interface, the interior surface of the plate hole is threaded, either with a single or multiple helical leads. The matching screw has a helical thread on the exterior surface of the screw head which mates with the thread on the inner surface of the hole. As the screw is inserted into the bone, the thread on the screw head begins to engage the thread in the hole. As the screw is tightened, increasing interference between the threads on the hole and the screw head eventually causes the screw to be “locked” with the plate. Unlike with a non-locking screw, the plate is generally not compressed against the bone by the locking screw.

The presently disclosed invention relates to bone plates having locking screw interfaces.

It will be readily observed that traditional bone plates having locking screw interfaces usually have screw holes with a generally cylindrical shape and an axis that is normal to the circular cross section of the hole. It is commonplace to use a locking screw with a threaded head that is inserted along the axis of the hole and whose shaft is therefore generally normal to the horizontal plane of the screw hole.

However, it is sometimes desirable to insert a locking screw at an angle that is off-axis, or not parallel to the axis of the hole. The traditional way that this is accomplished is by inserting the locking screw at the desired angle through the hole and then proceeding to “cross-thread” the screw's head into the thread in the hole. However, there are several drawbacks to this cross-threading technique. For one, because the threads on the screw head and the hole generally match, the screw will have the tendency to align itself with the axis of the hole, making it difficult to select a precise angle. This is particularly true when the desired screw angle is close to the angle of the axis of the hole.

In addition, since the thread of the screw is a helix and the thread in the hole of the plate is a helix, the engagement will be different and will have a different locking strength depending on the precise positional engagement of the two features. Although the threads of the screw and hole could be made to significantly differ, they would still engage each other inconsistently depending on the angle of the screw relative to the plate, and therefore result in significant variation in the strength of the interface depending on variations in the angle of engagement.

Other limitations include the required precision of the parts. With a threaded arrangement, if the parts are even slightly too big, they will protrude above the plate. In contrast, if the parts are just slightly too small, they will penetrate through the plate. This limitation leads to expensive machining and significant inspection to ensure proper functionality of the parts. Furthermore, these mechanical locking means often require dissimilar metals such as titanium and CoCr or relief cuts in the threaded hole that add cost and complexity. In some instances, the cross-threading results in burrs being cut that can lead to soft tissue irritation and even tendon rupture.

Therefore, there exists a need to provide a plate with a hole which facilitates the insertion of an off-axis locking bone screw without the drawbacks, such as cross-threading, apparent in other presently known methods.

### SUMMARY OF THE INVENTION

It is among the objects of this invention to overcome the limitations of the heretofore-known devices by providing inventive features to achieve: a.) insertion of an off-axis bone screw; b.) consistent locking strength between a locking screw and a plate; and c.) precision and stability in the angle of insertion of an off-axis bone screw.

It is an object of the present invention to provide an orthopedic implant adapted for fastening to a bone by use of a screw, the screw comprising an externally threaded head having a thread depth. In one embodiment in accordance with the present invention, the implant comprises a plate composed of a biocompatible material, and a hole through the plate defining an internal hole wall and having an innermost surface, the hole adapted to receive said screw. The internal hole wall of the hole comprises a primary inner width and a secondary outer width with a distance therebetween, wherein the distance between the primary inner width and the secondary outer width defines a volume. The internal hole wall further comprises a non-helical pattern. The volume defined between the primary inner width and the secondary outer width further comprises a biocompatible material and a plurality of blind holes extending from the primary inner width to the secondary outer width resulting in a porosity.

In at least one embodiment, between 30% and 70% of the volume defined by the primary inner width and the secondary outer width is comprised of the biocompatible material and the remaining 70% to 30% of the volume is devoid of any material; and wherein the internal hole wall comprises the non-helical pattern.

In another embodiment, the porosity of the biocompatible material within the volume defined by the primary inner width and the secondary outer width is between 0.3 and 0.7. In further embodiments, the distance between the primary inner width and the secondary outer width is at least one half the thread depth of the screw. In another embodiment, the biocompatible material of the internal hole wall is integral and indistinguishable from that of the biocompatible material of the plate.

In accordance with other embodiments of the present invention, the hole may have a cylindrical or non-cylindrical (e.g., oblong) shape.

In other embodiments, the plate further comprises a top side and bottom side, wherein the hole through the plate narrows from the top side to the bottom side of the plate, thereby creating a decreasing primary inner width. The decreasing primary inner width of the hole may result in a substantially frusto-conical profile in some embodiments.

In further embodiments, the plurality of blind holes may run partially or completely along a length of the internal hole wall. In embodiments with at least three of the plurality of blind holes, the blind holes may be evenly spaced. In other embodiments, the plurality of blind holes may be generally tapered to form a substantially conical pattern by the innermost surface of the internal hole wall.

In some embodiments, the non-helical pattern of the internal hole wall may comprise a plurality of curves formed by a plurality of ridges, the plurality of ridges intersecting to form the plurality of blind holes. In other embodiments, the non-helical pattern may comprise a plurality of crisscrossing diagonal lines forming a waffle pattern, the plurality of crisscrossing diagonal lines intersecting to form the plurality of blind holes. The non-helical pattern may alternatively comprise a plurality of rings interrupted by a slot, wherein the slot is adapted to receive an instrument, such as, for example, a bending iron. The non-helical pattern may also comprise a plurality of pockets. The plurality of pockets may form the plurality of blind holes. In some embodiments, the plurality of pockets may optionally be interrupted by a slot, wherein the slot is adapted to receive an instrument.

In at least one further embodiment, a space between each of the plurality of blind holes forms a crest width. The crest width may be between 0.125 to 0.5 millimeters.

In yet another embodiment, the hole may comprise at least one substantially straight side. In such embodiments, the non-helical pattern of the internal hole wall may comprise a plurality of parallel diagonal grooves along the at least one substantially straight side of the hole. The plurality of parallel diagonal grooves may form the plurality of blind holes.

Understanding that an area of the internal hole wall defined by the primary inner width and the secondary outer width is, optionally, not disposed on the surface, but instead is the actual plate material produced with a desired pore size, pore shape, and pore distribution; wherein the resulting porosity in the volume defined by the primary inner width and the secondary outer width results from the plurality of blind holes in the internal hole wall extending from the primary inner width to the secondary outer width; and wherein the internal hole wall comprises a non-helical pattern.

Although the invention is illustrated and described herein as embodied in several exemplary embodiments, it is nevertheless not intended to be limited to only the details shown, since various modifications and structural changes may be made therein without departing from the spirit of the invention and within the scope and range of equivalents of the claims.

The construction of the invention, however, together with additional objects and advantages thereof will be best understood from the following description of the specific disclosed embodiments when read in connection with the accompanying drawings.

### BRIEF DESCRIPTION OF THE DRAWINGS

FIGS. 1 and 2 illustrate cross-sectional isometric views of an orthopedic implant in accordance with one embodiment of the present invention.

FIG. 3 illustrates an isometric view of a locking screw in accordance with one embodiment of the present invention.

FIGS. 4 and 5 illustrate isometric views of a hole of an orthopedic implant with a hole having an oblong shape and a plurality of blind holes in accordance with another embodiment of the present invention.

FIG. 6 illustrates an isometric view of the orthopedic implant with the oblong shaped hole of FIGS. 4 and 5 having received a screw through each hole of the orthopedic implant in accordance with one embodiment of the present invention.

FIG. 7 illustrates a cross-sectional isometric view of an orthopedic implant with a “waffle” non-helical pattern in accordance with another embodiment of the present invention,

FIG. 8 illustrates a cross-sectional view of the orthopedic implant of FIG. 7 showing additional features in accordance with one embodiment of the present invention.

FIG. 9 illustrates an isometric view of a hole of an orthopedic implant having a hole with substantially straight sides and having a plurality of diagonal grooves forming a non-helical pattern in accordance with another embodiment of the present invention.

FIG. 10 illustrates a cross-sectional view of the hole of the orthopedic implant of FIG. 9 showing additional features in accordance with one embodiment of the present invention.

FIG. 11 illustrates an isometric view of the hole of the orthopedic implant of FIGS. 9 and 10 having received a screw through the hole of the orthopedic implant in accordance with one embodiment of the present invention.

FIGS. 12, 13 and 14 illustrate a cross-sectional isometric view, a top view, and a cross-sectional view, respectively, of a hole of an orthopedic implant having a plurality of pockets interrupted by a slot in accordance with another embodiment of the present invention.

FIGS. 15 and 16 illustrate top and bottom isometric views, respectively, of the orthopedic implant of FIGS. 12 through 14 having received a screw, a bending iron, and a drill guide through holes of the orthopedic implant in accordance with one embodiment of the present invention.

FIGS. 17 and 18 illustrate top and bottom isometric views, respectively of an orthopedic implant having received an off-axis drill guide through a hole of the orthopedic implant in accordance with another embodiment of the present invention.

FIG. 19 illustrates an isometric view of an orthopedic implant having a hole with substantially straight sides and having a plurality of pockets forming a non-helical pattern in accordance with another embodiment of the present invention.

FIG. 20 illustrates a cross-sectional view of the hole of the orthopedic implant of FIG. 19 showing additional features in accordance with one embodiment of the present invention.

FIGS. 21 and 22 illustrate an isometric view and a top view, respectively, of an orthopedic implant having a hole with substantially straight sides and having a plurality of pockets interrupted by a slot in accordance with another embodiment of the present invention.

FIG. 23 illustrates a cross-section view of the hole of the orthopedic implant of FIGS. 21 and 22 showing additional features in accordance with one embodiment of the present invention.

FIG. 24 illustrates an isometric view of a non-locking screw in accordance with one embodiment of the present invention.

FIGS. 25 and 26 illustrate isometric top and bottom views, respectively, of an orthopedic implant having a hole with substantially straight sides and having a plurality of diagonal grooves forming a non-helical pattern, the hole having received the non-locking screw of FIG. 24 in accordance with one embodiment of the present invention.

### DETAILED DESCRIPTION OF THE INVENTION

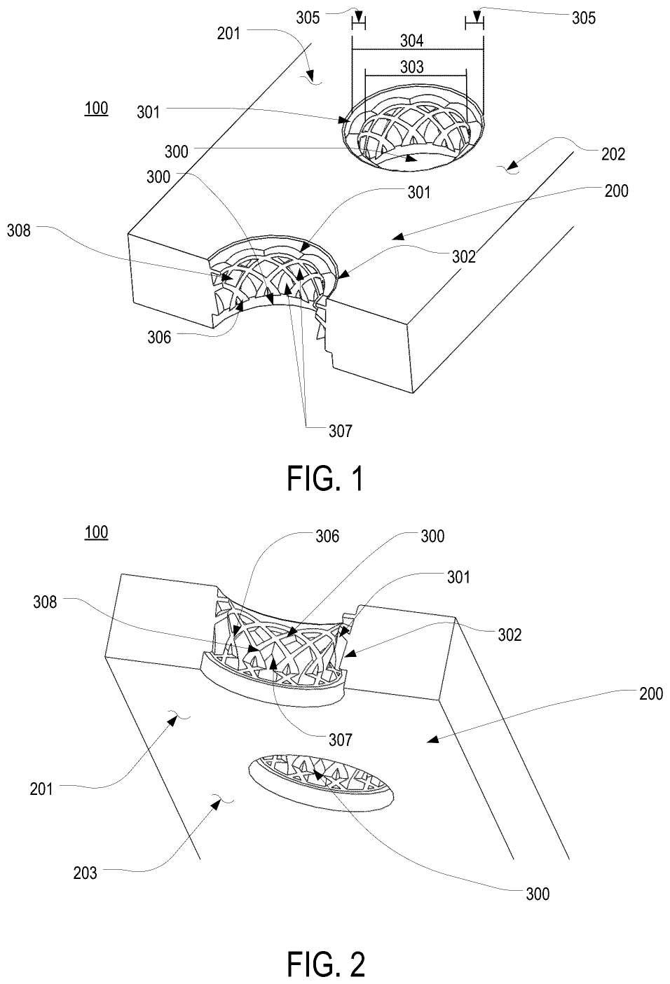

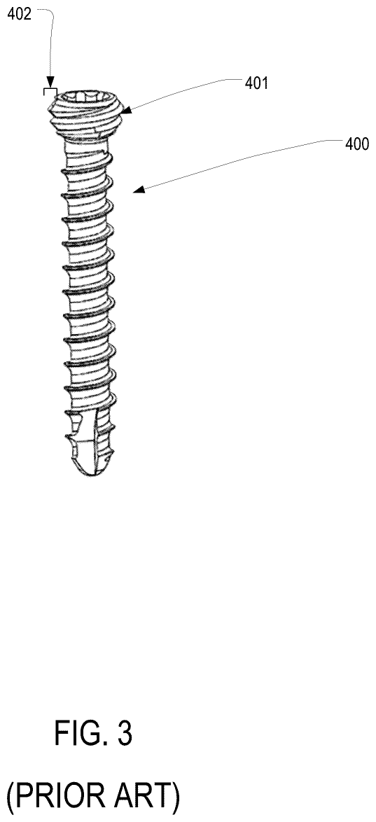

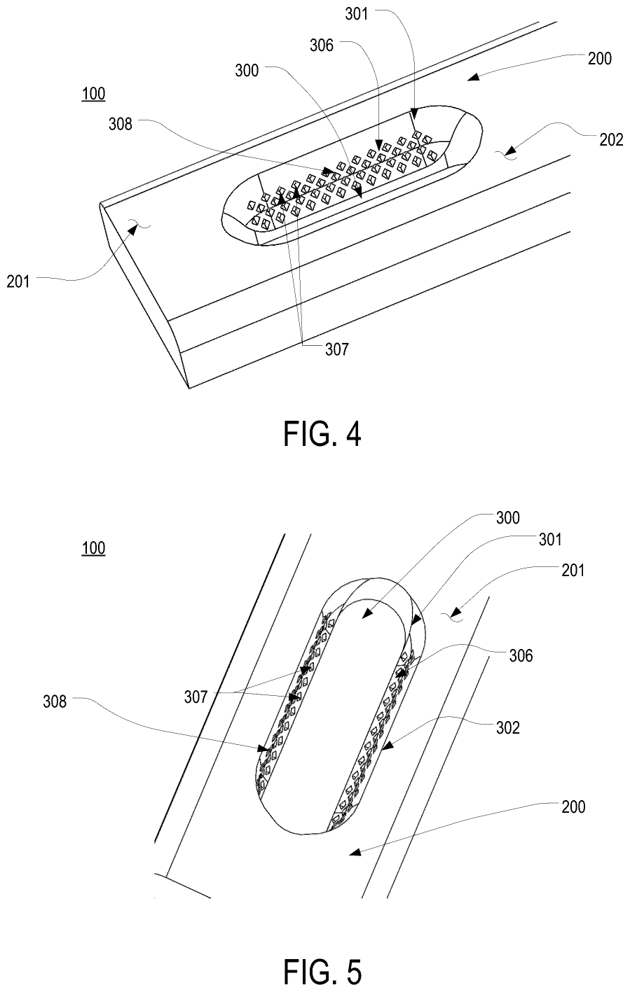

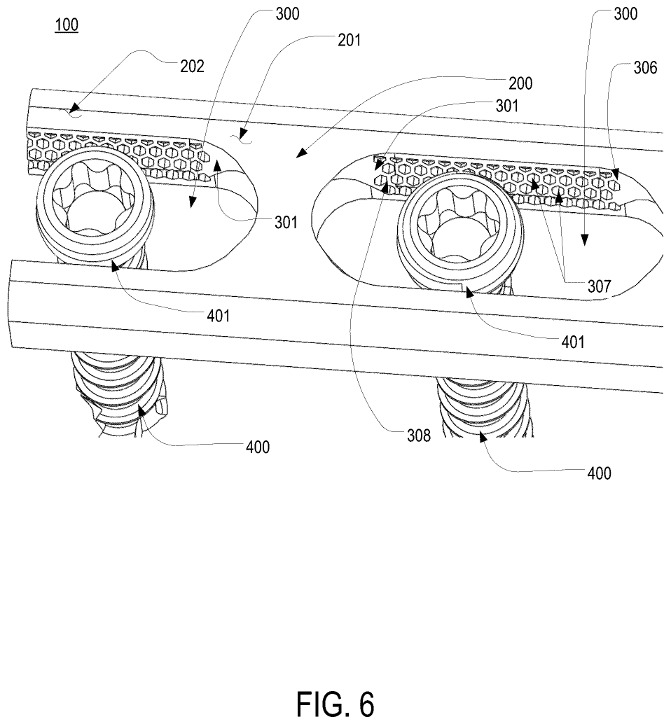

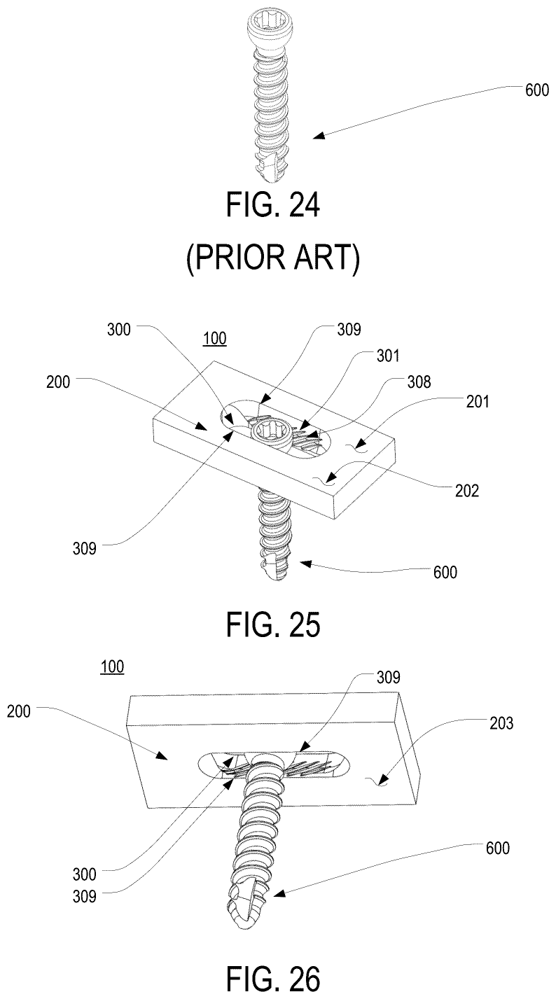

FIGS. 1 and 2 illustrate cross-sectional isometric views of one embodiment of an orthopedic implant (100) adapted for fastening to a bone by use of a screw (400), the screw (400) comprising an externally threaded head (401) having a thread depth (402) (see FIG. 3), in accordance with the present invention. The implant (100) comprises a substantially flat plate (200) composed of a biocompatible material (201), and a hole (300) through the plate (200) defining an internal hole wall (301) and having an innermost surface (302). It should be noted that an implant (100) in accordance with the present invention may comprise one or more holes (300) (see, for example, FIGS. 1, 2, 6, 7, 12, and 15-18 for embodiments with more than one hole (300)). Referring again to FIGS. 1 and 2, shown are cross-sectional views of a hole (300) in accordance with one embodiment of the present inventions. The hole (300) is adapted to receive said screw (400) (see FIGS. 6, 11, 15 and 16). An exemplary screw (400) is shown in FIG. 3. The screw (400) may comprise an externally threaded head (401) with a thread depth (402). The screw depicted in FIG. 3 is a locking screw (400). Various views of other holes (300) in accordance with various embodiments of the present invention are shown in FIGS. 4-23 and 25-26. FIG. 24 depicts a non-locking screw (600) without a threaded head.

Referring to FIGS. 1 and 8, the internal hole wall (301) of the hole (300) further comprises a primary inner width (303) and a secondary outer width (304) with a distance (305) therebetween. The distance (305) resulting between the primary inner width (303) and the secondary outer width (304) of the internal hole wall (301) defines a volume. Referring again to FIGS. 1, 4, 5, 7 and 8, the volume further comprises a biocompatible material (306) and a plurality of blind holes (307) extending from the primary inner width (303) to the secondary outer width (304) resulting in a porosity. For clarity purposes, only a couple of the plurality of blind holes (307) are referenced in each of the figures. The reference characters pointing to the plurality of blind holes (307) are demonstrative only as several blind holes (307) are illustrated throughout the figures. The internal hole wall (301) further comprises a non-helical pattern (308). The plurality of blind holes (307) may form the non-helical pattern (308) of the internal hole wall (301).

Referring again to FIGS. 1 and 8, in one embodiment, the distance (305) between the primary inner width (303) and the secondary outer width (304) is at least one half the thread depth (402) (shown in FIG. 3) of the screw (400).

In other embodiments, the plate (200) further comprises a top side (202) and a bottom side (203) (see FIGS. 1, 2, 8 and 10), wherein the hole (300) through the plate (200) narrows from the top side (202) to the bottom side (203) of the plate (200) creating a decreasing primary inner width (303) (see, for example, FIG. 8). The decreasing primary inner width (303) of the hole (300) may result in a substantially frusto-conical profile in some embodiments. In at least one embodiment in accordance with the present invention, the angulation may be 4 to 10 degrees per side, for a total angulation of 8 to 20 degrees between both sides.

In at least one embodiment in accordance with the present invention, between 30% and 70% of the volume defined by the primary inner width (303) and the secondary outer width (304) is comprised of the biocompatible material (306) and the remaining 70% to 30% of the volume is devoid of any material. The volume devoid of any material may be formed by the plurality of blind holes (307).

In further embodiments, the porosity of the biocompatible material (306) within the volume defined by the primary inner width (303) and the secondary outer width (304) is between 0.3 and 0.7. Porosity represents the ratio of the volume of void space to the overall volume.

It should be noted that in some embodiments, the biocompatible material (306) of the volume of the internal hole wall (301) may be integral and indistinguishable from that of the biocompatible material (201) of the plate (200).

Referring now to FIGS. 4 through 6, shown are various views of an implant (100) with a hole (300) having an oblong shape in accordance with at least one embodiment of the present invention. Shown in FIGS. 4 through 6 is a first embodiment of an implant (100) of the present invention comprising a substantially flat plate (200) having a hole (300) therethrough, the hole (300) defining an internal hole wall (301). As depicted in the embodiment of FIGS. 4 through 6, the hole (300) may have an oblong shape. The internal hole wall (301) of the hole (300) further comprises a plurality of blind holes (307) which may be evenly spaced and run partially (shown) or completely (not shown) along a length of the internal hole wall (301) of the hole (300). As depicted in the embodiment of FIGS. 4 through 6, the plurality of blind holes may each be diamond or hexagonal in shape. However, other shapes (e.g., circle, oval, square, rectangle, octagon, etc.) are possible in further embodiments of the present invention. The plurality of blind holes (307) may be generally tapered to form a generally conical pattern by the innermost surface (302) of the internal hole wall (301). The taper ensures that as a screw (400) is inserted through the hole (300), greater resistance and locking force is progressively generated.

Referring back to FIGS. 1 and 2, shown are various views of an implant (100) with a hole (300) having a circular shape in accordance with at least one embodiment of the present invention. Shown in FIGS. 1 and 2 is a second embodiment of the present invention, wherein the implant (100) comprises a substantially flat plate (200) having a hole (300) therethrough with a circular shape. In the depicted embodiment of FIGS. 1 and 2, the non-helical pattern (308) of the internal hole wall (301) of the hole (300) comprises a plurality of curves formed by a plurality of ridges (relative to the surface plane of the hole (300)) that progressively narrow to create a substantially conical profile. The plurality of ridges intersect resulting in a plurality of blind holes (307) that extend from the internal hole wall (301) of the hole (300) to the innermost surface (302) of the hole (300).

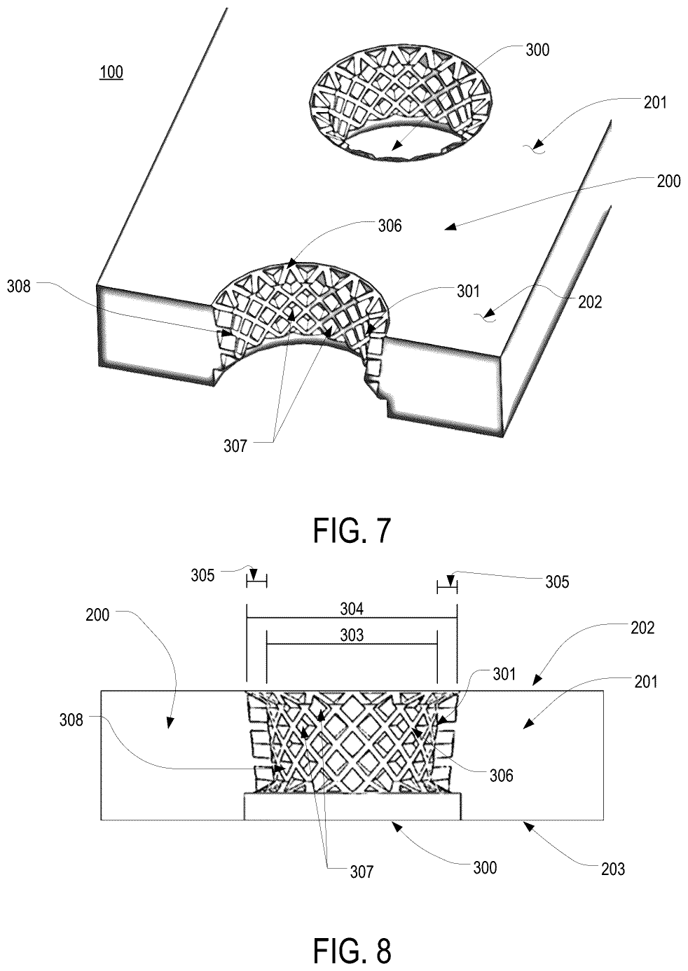

Referring now to FIGS. 7 and 8, shown are perspective and cross-sectional views of an implant (100) with a hole (300) having a circular shape in accordance with another embodiment of the present invention. Shown in FIGS. 7 and 8 is a third embodiment of the present invention, wherein the implant (100) comprises a substantially flat plate (200) having a hole (300) with a circular shape therethrough. In the depicted embodiment of FIGS. 7 and 8, the internal hole wall (301) of the hole (300) comprises a plurality of crisscrossing diagonal lines which form a “waffle” or “basket” non-helical pattern (308). The plurality of crisscrossing diagonal lines may intersect to form the plurality of blind holes (307). The hole (300) can, as shown, define a series of conical sections of various slopes. In the example shown in FIGS. 7 and 8, there is an initial highly sloped section to ease centering of the screw head (401), an intermediate slightly conical section to facilitate progressive tightening of the head (401) of the screw (400) as it is inserted into the hole (300).

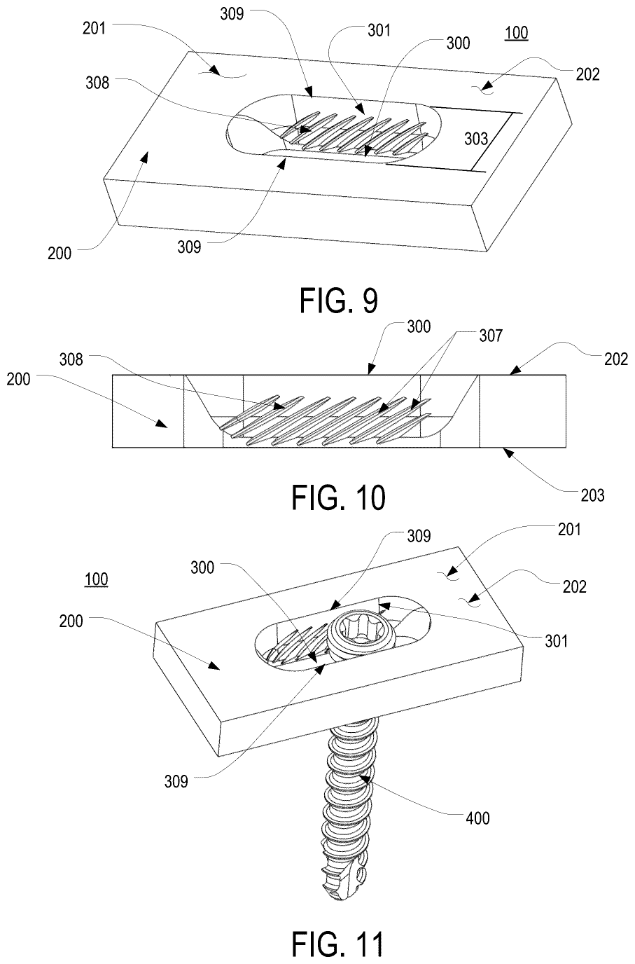

Referring next to FIGS. 9 and 10, shown are perspective and cross-sectional views, respectively, of an implant (100) with a hole (300) in accordance with another embodiment of the present invention. FIG. 11 shows the implant (100) with the hole (300) of FIGS. 9 and 10 having received a screw (400). Shown in FIGS. 9 through 11 is a fourth embodiment of the present invention, wherein the implant (100) comprises a substantially flat plate (200) having a hole (300) therethrough, the hole (300) having a slot shape and having at least one substantially straight side (309). The embodiment shown has two substantially straight sides (309). In the depicted embodiment, the internal hole wall (301) of the hole (300) has a plurality of parallel diagonal grooves along each of the at least one substantially straight side (309) of the slot-shaped hole (300) that form the non-helical pattern (308). The plurality of parallel diagonal grooves may form the plurality of blind holes (307) (see FIG. 10). The slot-shaped hole (300) optionally narrows down from the top side (202) to the bottom side (203) of the plate (200) to create a progressively smaller primary inner width (303) (FIG. 9) opening and gradually increase the locking force as a screw (400) is introduced.

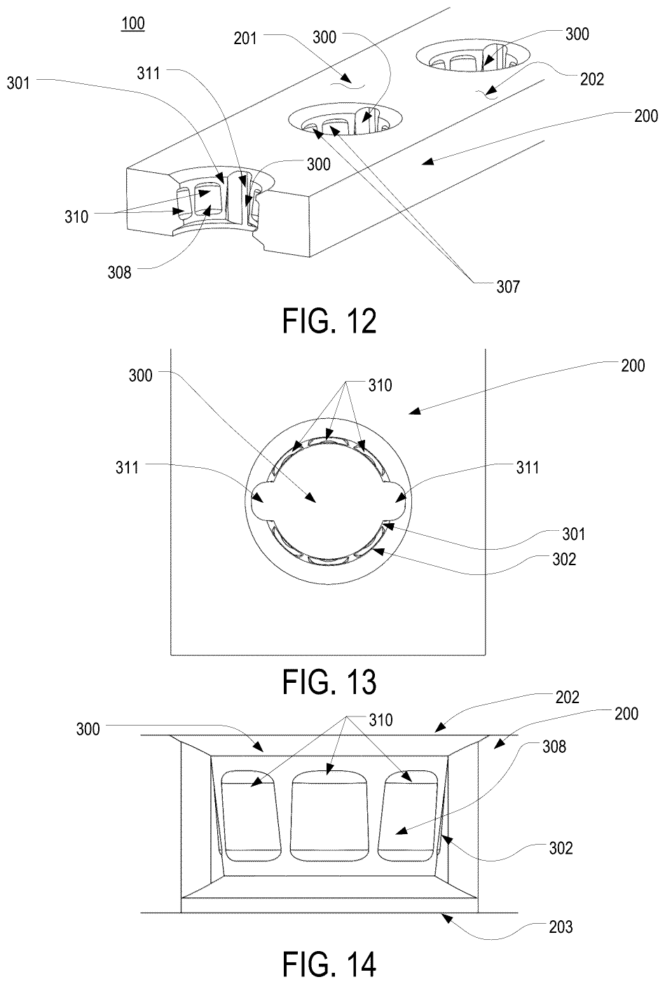

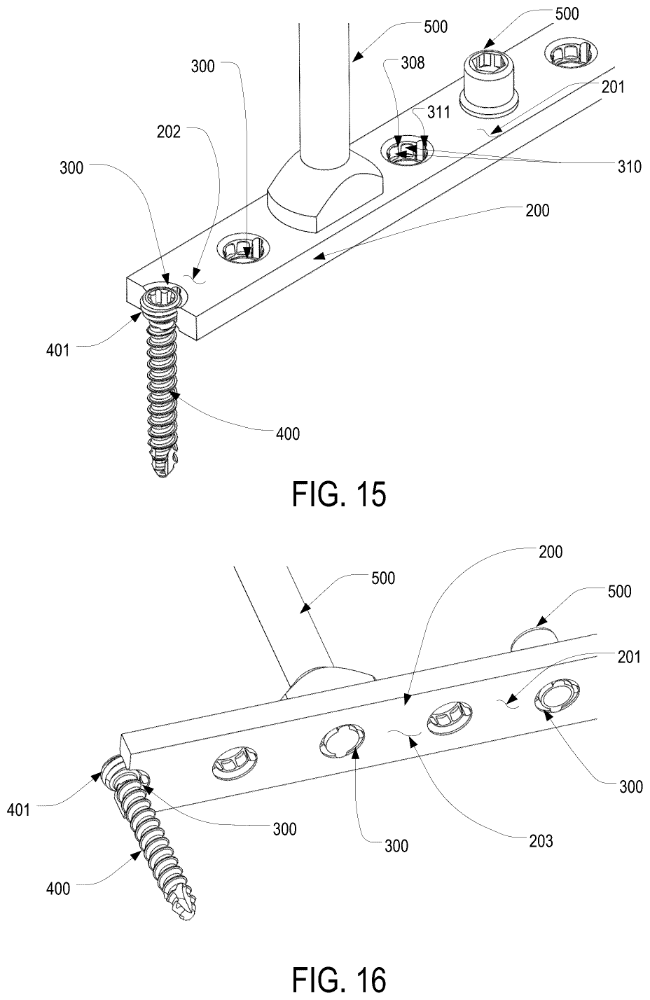

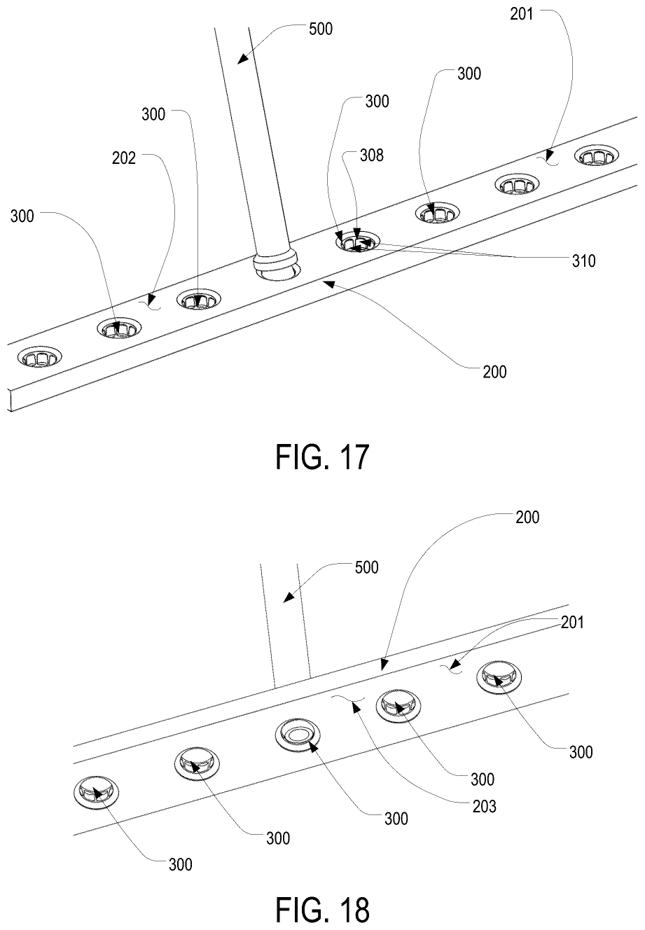

Now referring to FIGS. 12 through 16, shown are various views of an implant (100) with a hole (300) having a circular shape in accordance with yet another embodiment the present invention. Shown in FIGS. 12 through 16 is a fifth embodiment, wherein the non-helical pattern (308) of the internal hole wall (301) comprises a plurality of pockets (310) that are interrupted by a slot (311). The plurality of pockets (310) may form the plurality of blind holes (307) (see FIG. 12). The slot (311) can be adapted to receive various instruments (500), such as for example, a pre-assembled straight-axis guide or a bending iron as shown in FIGS. 15 and 16, or an off-axis guide as shown in FIGS. 17 and 18. The slot (311) can be used, for example, to facilitate the installation of pre-assembled guides, while also allowing bending irons to be quickly installed. The innermost surface (302) of the hole (300) is able to support the loads from a non-locking screw (600) (see FIG. 24) and the variable angle locking screw (400) (shown in FIGS. 15 and 16) is able to create an interface that can support significant bending loads.

Referring again to FIGS. 12 through 14, the plurality of pockets (310) may optionally have a concave shape and may run vertically, extending from the top side (202) of the plate (200) and along the internal hole wall (301) of the hole (300) down toward the bottom side (203) of the plate (200). In the embodiment shown, the plurality of pockets (310) extends from the top side (202) of the plate (200) along the internal hole wall (301) to a partial depth of the hole (300) (see FIG. 14). In some embodiments, the plurality of pockets (310) does not extend all the way through to the top side (202) of the plate (200). In other embodiments, the plurality of pockets (310) does not extend all the way through to the bottom side (203) of the plate. In further embodiments, such as the one depicted, the plurality of pockets (310) does not extend all the way through to the top side (202) nor all the way through to the bottom side (203) of the plate (200), but instead has a distance between the plurality of pockets and the top side (202) of the plate (200) and a distance between the plurality of pockets (310) and the bottom side (203) of the plate (200). In such embodiments, the plurality of pockets (310) is adapted to not break through the top side (202) or the bottom side (203) of the plate (200) (as shown in the depicted embodiment). Referring further to FIG. 14, the plurality of pockets (310) extending along only a partial depth of the hole (300), and not through to the bottom side (203) of the plate (200), facilitates the progressive tightening of the head (401) of the screw (400) as it is inserted into the hole (300) (see FIGS. 15 and 16).

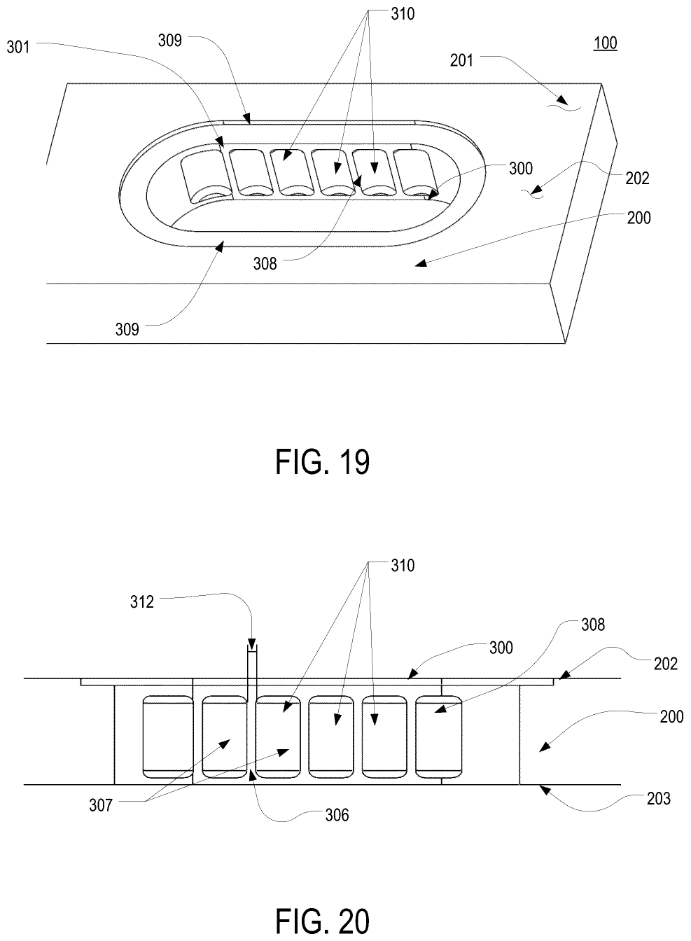

Referring next to FIGS. 19 and 20, shown are perspective and cross-sectional views, respectively, of a sixth embodiment of the present invention, wherein the implant (100) comprises a substantially flat plate (200) having a hole (300) therethrough. As depicted in FIG. 19, the hole (300) may have a slot shape having at least one substantially straight side (309). In the embodiment shown, the hole (300) has two substantially straight sides (309). Furthermore, as shown in the depicted embodiment of FIGS. 19 and 20, the internal hole wall (301) of the hole (300) has a plurality of plurality of pockets (310) along each of the at least one substantially straight side (309) of the slot-shaped hole (300) that form the non-helical pattern (308).

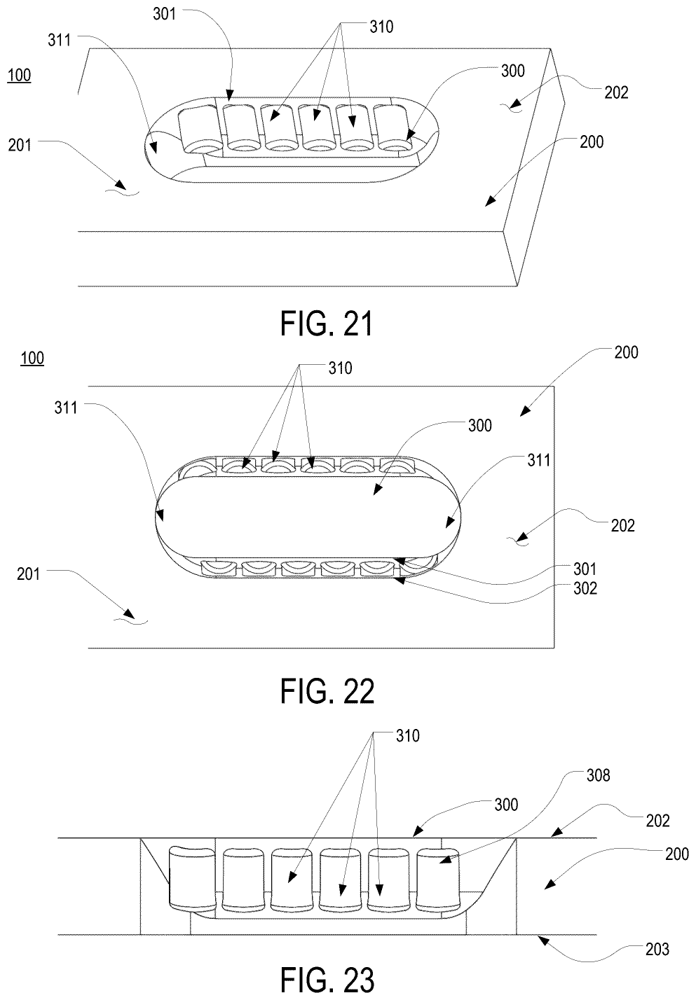

Referring again to FIG. 20, the plurality of pockets (310) may optionally have a concave shape and may run vertically, extending from the top side (202) of the plate (200) down toward the bottom side (203) of the plate (200). Referring to FIG. 19, the plurality of pockets (310) may extend from the top side (202) of the plate (200) along the internal hole wall (301) to a partial depth of the hole (300). In some embodiments, the hole (300) may further comprise a lip or ridge that prevents the screw (400) from penetrating through the plate (200). The lip may be located at the opening of the hole (300) on the top side (202), and optionally the bottom side (203), of the plate (200). Referring now to FIGS. 21 through 23, the plurality of pockets (310) may optionally be interrupted by a slot (311). The slot may be adapted to contact the head (401) of a non-locking screw (600) (see FIGS. 25 and 26) to force the plate (200) to move along a long axis of the plate (200) for the purpose of dynamic compression during installation.

Referring back to FIGS. 19 and 20, in further embodiments in accordance with the present invention, the space of the internal hole wall (301) (FIG. 19) between successive peaks of each of the plurality of blind holes (307), depicted as a plurality of pockets (310) in FIGS. 19 and 20, comprises a crest width (312) (FIG. 20). The crest width (312) may comprise the biocompatible material (306) (FIG. 20) of the internal hole wall (301) (FIG. 19). The crest width (312) is adapted to be narrow enough to permit a screw (400) (not shown) to displace the biocompatible material (306) of the internal hole wall (301) to be displaced, while thick enough to secure the screw (400). In at least some embodiments, the crest width (312) may be between 0.125 to 0.5 millimeters. The crest width (312) depicted in the embodiment of FIG. 20 is a representative example only as any embodiment in accordance with the present invention may comprise a crest width (312) between the plurality of blind holes (307).

It should be apparent that many designs can be conceived that are able to satisfy the various characteristics of the present invention. For example, a given surface area of the internal hole wall (301) may correspond to a minimum that can support the compressive forces of a non-locking screw (600) (see FIG. 24 for an example of a non-locking screw). For example, FIGS. 25 and 26 illustrate another embodiment in accordance with the present invention wherein the hole (300) is adapted to receive a screw, and wherein the screw comprises a non-locking screw (600). The implant (100) may also include a reduced strength area to form the interface that will allow a locking screw (400) to deform the surface of the internal hole wall (301). Additionally, a region in the hole (300) may be weakened through a designed porosity that results in lowering the density of the volume of the material that is contacted to form the interface between the locking screw (400) and the plate (200). In some embodiments, the hole (300) may comprise a lip or ridge that prevents the screw (400) from penetrating through the plate (200). In other embodiments, a slot (311) or interruption on the internal hole wall (301) can be used to install drill guides and instruments that can be used to install the plate (200) (see, for example, the embodiment in FIGS. 15 and 16). Also, the hole (300) may be sized to interact with an off-axis drill guide to establish the direction of the drill and the limit of the angle that the screw (400) can be installed in the plate (200) (see, for example, the embodiment in FIGS. 17 and 18), wherein the limits are defined as a function of the off-axis drill interacting with the opening of the hole (300) at the top side (202) of the plate (200) to limit articulation.

In some embodiments, the hole (300) may have a cylindrical, conical, or spherical shape. Other embodiments, such as those using holes (300) having an oblong (see FIGS. 4-6) or a slot (see FIGS. 9-11) shape, illustrate that the patterns can be applied to hole (300) shapes that are not cylindrical, conical, or spherical. It should be noted that both locking screws (400) (see FIG. 3) and non-locking screws (600) (see FIG. 24) can be inserted into the hole (300). The surface area of the internal hole wall (301) is able to support the loads of the non-locking screw (600) as it compresses the implant onto the bone, while the locking screw (400) is able to cut into the non-helical pattern (308) of the internal hole wall (301) to ensure that the fastener does not back out. The added benefit of being able to support a bending load is also a benefit when using a locking screw (400).

In the above embodiments, it will be observed that between 30% and 70% of the volume between the primary inner width (303) and the secondary outer width (304) of the internal hole wall (301) has been removed in order to form the various non-helical patterns (308) that are engaged by the locking screw (400). This results in an overall porosity of the engagement area between 0.3 and 0.7.

In the above embodiments, to facilitate insertion of the screw (400), the material of the internal hole wall (301) may be softer than the material of the threads on the screw head (401).

Although described above in connection with particular embodiments, these descriptions are not intended to be limiting, as other plates and holes can be made in accordance with the description herein, but of different size or scale, as desired. As such, although the invention is illustrated and described herein, various modifications and structural changes may be made therein without departing from the spirit of the invention and within the scope and range of equivalents of the claims.

Any reference in this specification to “one embodiment,” “an embodiment,” an “example embodiment,” etc., means that a particular feature, structure, or characteristic described in connection with the implementation is included in at least one embodiment of the invention. The appearances of such phrases in various places in the specification are not necessarily referring to the same embodiment. In addition, any elements or limitations of any invention or embodiment thereof disclosed herein can be combined with any and/or all other elements or limitations (individually or in any combination) or any invention or embodiment thereof disclosed herein, and all such combinations are contemplated with the scope of the invention without limitation thereto.

Although described above in connection with particular configurations, these descriptions are not intended to be limiting as various modifications may be made therein without departing from the spirit of the invention and within the scope and range of equivalents of the described embodiments. Encompassed embodiments of the present invention can be used in all plates calling for a non-helical fastener hole.

While a number of embodiments of the present invention have been described, it is understood that these examples and embodiments are illustrative only, and not restrictive, and that many modifications may become apparent to those of ordinary skill in the art and are to be included within the spirit and purview of this application. For example, any element described herein may be provided in any desired size (e.g., any element described herein may be provided in any desired custom size or any element described herein may be provided in any desired size selected from a “family” of sizes, such as small, medium, large). Further, one or more of the components may be made from many different suitable materials.

In addition, various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as within the scope of the claims, together with all equivalents thereof.

What is claimed is:

1. An orthopedic implant adapted for fastening to a bone by use of a screw, the screw comprising an externally threaded head having a thread depth, the implant comprising:
a plate composed of a biocompatible material;
a hole through the plate defining an internal hole wall and having an innermost surface, the hole adapted to receive said screw;
the internal hole wall comprising a primary inner width and a secondary outer width with a distance therebetween, wherein the distance resulting between the primary inner width and the secondary outer width of the internal hole wall defines a volume;
the volume comprising a biocompatible material and a plurality of blind holes extending from the primary inner width to the secondary outer width resulting in a porosity;
wherein the internal hole wall comprises a non-helical pattern.

2. The implant of claim 1 wherein between 30% and 70% of the volume defined by the primary inner width and the secondary outer width is comprised of the biocompatible material and the remaining 70% to 30% of the volume is devoid of any material.

3. The implant of claim 1 wherein the porosity of the biocompatible material within the volume defined by the primary inner width and the secondary outer width is between 0.3 and 0.7.

4. The implant of claim 1 wherein the distance between the primary inner width and the secondary outer width is at least one half the thread depth.

5. The implant of claim 1 wherein the biocompatible material of the volume of the internal hole wall is integral and indistinguishable from that of the biocompatible material of the plate.

6. The implant of claim 1 wherein the hole has a non-cylindrical shape.

7. The implant of claim 6 wherein the hole has an oblong shape.

8. The implant of claim 1 wherein the hole has a cylindrical shape.

9. The implant of claim 1 wherein the plate comprises a top side and a bottom side, and wherein the hole through the plate narrows from the top side to the bottom side of the plate creating a decreasing primary inner width.

10. The implant of claim 1 wherein at least three of the plurality of blind holes are evenly spaced.

11. The implant of claim 1 wherein the plurality of blind holes runs partially along a length of the internal hole wall.

12. The implant of claim 1 wherein the plurality of blind holes runs completely along the length of the internal hole wall.

13. The implant of claim 1 wherein the plurality of blind holes generally taper to form a substantially conical pattern by the innermost surface of the internal hole wall.

14. The implant of claim 1 wherein the non-helical pattern of the internal hole wall comprises a plurality of curves formed by a plurality of ridges, the plurality of ridges intersecting to form the plurality of blind holes.

15. The implant of claim 1 wherein the non-helical pattern of the internal hole wall comprises a plurality of crisscrossing diagonal lines forming a waffle pattern, the plurality of crisscrossing diagonal lines intersecting to form the plurality of blind holes.

16. The implant of claim 1 wherein the hole comprises at least one substantially straight side.

17. The implant of claim 16 wherein the non-helical pattern of the internal hole wall comprises a plurality of parallel diagonal grooves forming the plurality of blind holes along the at least one substantially straight side of the hole.

18. The implant of claim 1 wherein a space between each of the plurality of blind holes forms a crest width between 0.125 to 0.5 millimeters.

19. The implant of claim 1 wherein the non-helical pattern defined by the internal hole wall comprises a plurality of pockets forming the plurality of blind holes.

20. The implant of claim 19 wherein the plurality of pockets is interrupted by a slot adapted for receiving an instrument.
